(19) United States
(12) Patent Application Publication
(10) Pub. No.: US 2009/0155318 A1
(43) Pub. Date: Jun. 18, 2009

(54) PROCESS TO FORM NANO-SIZED MATERIALS, THE COMPOSITIONS AND USES THEREOF

(76) Inventor: Howard W. H. Lee, Saratoga, CA (US)

Correspondence Address: PETERS VERNY , L.L.P., 425 SHERMAN AVENUE, SUITE 230, PALO ALTO, CA, 94306, US

(21) Appl. No.: 11/933,397

(22) Filed: Oct. 31, 2007

Related U.S. Application Data
(60) Provisional application No. 60/855,892, filed on Oct. 31, 2006.

(51) Int. Cl. A61K 8/19; A61K 33/00

(52) U.S. Cl. 424/401; 424/718; 977/919

(57) ABSTRACT

Various embodiments of the invention include systems and methods of generating nano-sized salt particles. These methods include generation of a supersaturated solution of a salt MX by mixing of salts MY and NX in which the solubility of MX is lower than that of MY, NX or NY. In some embodiments, nano-sized particles of MX are grown further by further adiabatic addition of salts MY and/or NX. In some embodiments, MX includes potassium nitrate. Some embodiments include compositions of MX configured for use in treatment of dentine sensitivity.

### CROSS-REFERENCE TO RELATED APPLICATIONS

This application claims benefit of U.S. Provisional Patent Application No. 60/855,892 filed Oct. 31, 2006 and entitled “Process to form Nanostructured Materials the Compositions and Uses Thereof.” The disclosure of this application is herein incorporated by reference.

### BACKGROUND OF THE INVENTION

1. Field of the Invention

This invention relates to methods and compositions to produce nano-sized materials having a defined size range. It includes the uses of these nano-sized materials. Specifically, nano-sized materials, e.g., potassium nitrate, and more specifically nano-sized particles of potassium nitrate, are used in dentistry to treat dentin hypersensitivity.

2. Related Art

### Background

Salts, for example ionic salts, are commonly used as components in a wide variety of products and industries, for example as components in medical and dental compositions. These components take a variety of forms, including, but not limited to, gels, solutions, suspensions, emulsions, and the like. In many applications the salts play an active roll in the function of the composition. In these applications the size of the salt particles plays an important roll in the compositions efficacy, and compositions that contain salt particles of a specific size, or particles within a specific size range, have improved function. For example, creating particles of smaller size, for example nano-sized particles, increases the surface area of the particles increasing the rate at which the particles participate in chemical reactions. Nano-sized particles are easier to suspend in solutions and create suspensions and/or emulsions that have increased stability. Nano-sized particles have characteristics that are related to particle size, such as color, which can be controlled through the control of the particle size, as opposed to through the addition of other substances, for example pigments. Compositions that utilize nano-sized salt particles are not currently taught or suggested by the existing art. Similarly, methods that allow the creation of salt particles of a specific size, or within a specific size range, are not discussed by the conventional art.

Examples of salt particles include potassium nitrate, and materials as found in U.S. Pat. No. 6,524,558. Fluoride compounds (fluoride therapies) promote the remineralisation of teeth, making teeth harder and more resistant to the formation of tooth decay, inhibiting oral bacteria's ability to create acids. Examples of fluoride compounds and several exemplary concentration ranges or example concentrations include sodium fluoride, for example, sodium monofluorophosphate (MFP), from about 225 ppm to about 22,500 ppm, acidulated phosphate fluoride (APF) from about 200 ppm to about 12,300 ppm, stannous fluoride from about 900 ppm to about 1500 ppm, for example 960 ppm and 1512 ppm, tin(II) fluoride (SnF2), amine fluorides (e.g., OLAFLUR® (N′-octadecyltrimethylenediamine-N,N,N′-tris(2-ethanol)-dihydrofluoride), DECTAFLUR® (9-octadecenylamine-hydrofluoride)), about 1000 ppm difluorsilane, and calcium fluoride. Examples of peroxy compounds are hydrogen peroxide, carbamide peroxide, calcium carbonate peroxide, sodium carbonate peroxide, sodium perborate (monohydrate or tetrahydrate), sodium percarbonate), and combinations thereof.

Dentin hypersensitivity is a common condition of transient tooth pain caused by a variety of external stimuli. The condition affects more than 40 million people in the United States annually. External stimuli that typically initiate the pain include thermal (cold), tactile (touch), or osmotic changes (sweets or drying the surface). While extreme stimuli can cause pain in all teeth, normal or otherwise, the hypersensitivity condition relates to a painful response to stimuli that does not normally produce pain. The specific response to stimuli varies among individuals due to differences in pain tolerance, environmental factors, and emotional state.

The primary underlying clinical cause for dentin hypersensitivity is exposed dentinal tubules. This allows fluid to flow within the tubules (according to the hydrodynamic theory) and impress on the nerves within the tubules, thus creating pain. The only hypersensitivity not associated with this etiology is the transient spontaneously resolving hypersensitivity associated with the dental bleaching process.

The most common clinical cause for exposed dentinal tubules is gingival recession. The recession may or may not be associated with bone loss. If bone loss occurs, then more dentinal tubules are exposed. When gingival recession occurs, root surfaces and cementum are exposed. Cementum, which is a very thin outer protective layer on dentin, is easily abraded or eroded away. This exposes to external stimuli the underlying dentin, which consists of tubules that contain the protoplasmic projections of the odontoblasts within the tooth pulp chamber. These cells contain nerve endings which, when disturbed by changes in tubular fluid pressure, depolarize or discharge, resulting in pain.

While not wanting to be bound by any particular theory, several theories have been proposed to explain the mechanism of dentin hypersensitivity. These include: (1) transducer theory; (2) modulation theory; (3) gate control and vibration theory; and (4) hydrodynamic theory. The most widely accepted theory for the origin of the pain is Brännström's hydrodynamic theory of dentin hypersensitivity. According to this theory, temperature changes or physical osmotic or pressure changes disturb fluids within the tubules. The resulting changes or movement of the tubular fluid stimulates pressure sensitive nerve receptors (baroreceptors) that give rise to neural discharge or depolarization. However, regardless of the origin or mechanism of the pain, the documented clinical condition that leads to hypersensitivity is exposed dentinal tubules.

Hypersensitivity is resolved without treatment or may require several weeks of desensitizing agents before results are observed. There are three principal treatment strategies: (1) cover the dentinal tubules with gingival grafts or dental restorations, (2) occlude the dentinal tubules preventing fluid flow, or (3) desensitize the nerve. All current treatments use some form or combinations of these three options.

However, no single agent or form of treatment is universally effective. Solutions, gels and pastes-containing fluorides in varying compounds and percentages, calcium hydroxide, strontium chloride, potassium nitrate, sodium citrate, formaldehyde, or potassium or ferric oxalate have varying degrees of success.

Currently potassium nitrate is the only material that can claim to desensitize the nerve. All over-the-counter desensitization dental products with ADA and FDA approval currently contain potassium nitrate. It is believed that potassium ions penetrate through the dentinal tubules to the nerve. The potassium ion then depolarizes the nerve and prevents repolarization, and thus precludes the transmission of pain signals to the brain. Currently, desensitizing toothpastes with ADA approval are Crest® Sensitivity Protection Fluoride Toothpaste, Orajel® Sensitive Pain Relieving Toothpaste for Adults, Colgate®; Sensitive Maximum Strength Toothpaste, and Protect®; Sensitive Teeth Gel Toothpaste.

None of the treatments described hereinabove provides a completely satisfactory remedy for pain.

### Relevant Literature

The following U.S. Pat. Nos. teaches the use of salts and anions to prevent caries, treat dental sensitivity and calcify dental tissues: 5,762,911, 6,217,851, 6,524,558, and 6,436,370.

### BRIEF SUMMARY OF THE INVENTION

Various embodiments of the present invention comprise a method for the controlled production of nano-sized particles by size of organic and inorganic salts, which method comprises: adding a salt MY to a solvent comprising NX and a coordinating species under non-adiabatic conditions concurrent with rapid stirring of the resulting solution which results in a supersaturated solution of salt MX and NY, concurrently maintaining said solution under non-adiabatic conditions to concentration, time, stirring, temperature and pressure which promote the formation of seed nano-sized particles of MX, followed by; Increasing the size of the nano-sized particles of MX to a specific size by further controlled addition of MY and NX under adiabatic conditions and by varying the amount and rate of addition on MY and NX under appropriate concentration, time, stirring, temperature and pressure conditions which do not create a second supersaturated state; Stopping the addition of MY to stop the growth of the formed nano-sized particles of MX; and Separating the nano-sized particles of MX having a specific size range from NY, wherein M is an organic or inorganic cation and Y is an anion and N is an organic or inorganic cation and X is an anion, and the coordinating species is selected from the group consisting of surfactants micelles and inverse micelles.

Various embodiments of the present invention comprise a method for the production of nano-sized particles of a salt MX, the method comprising: mixing reactant salts MY and NX in a solvent, in the presence of a soft template, chosen such that the solubilities of salts MY, NX and NY in S1 are larger than the solubility of salt MX in S1 at a reaction temperature; forming the nano-sized particles of salt MX by nucleation under supersaturated conditions; and separating the nano-sized particles of salt MX from the solvent.

Various embodiments of the present invention comprise a method for the size controlled production of nano-sized salt particles, the method comprising: adding a salt MY to a solvent comprising NX and a coordinating species, under non-adiabatic conditions; mixing the resulting solution to generate a supersaturated solution of salt MX and NY, maintaining said solution under non-adiabatic conditions to promote the nucleation of seed nano-sized particles of MX; increasing the size of the seed nano-sized particles of MX to a specific size range by further controlled addition of MY and NX under adiabatic conditions and by varying the amount and rate of addition on MY; stopping the addition of MY so as to stop the growth of the formed nanoparticles of MX; and separating the nano-sized particles of MX having a specific size range from NY, wherein M is a first organic or inorganic cation, Y is a first anion. N is a second different organic or inorganic cation, and X is a second different anion.

Various embodiments of the present invention comprise a method of treating dentin sensitivity, which method comprises: applying to the dentin nano-sized particles in a therapeutically effective amount in a pharmaceutically acceptable carrier; and allowing the nano-sized particles to penetrate tubules of the dentine.

Various embodiments of the present invention comprise a composition for the production of nano-sized particles, which composition comprises: a supersaturated solution of a salt MX, M including an organic or inorganic cation non-adiabatically introduced to the solution as a salt MY, X including an organic or inorganic anion non-adiabatically introduced to the solution as a salt NX, the supersaturated solution being at a temperature of less than 45° C.; and a soft template configured for controlling the formation of nano-sized particles of the salt MX.

Various embodiments of the present invention comprise a composition comprising: nano-sized particle of a salt MX configured to penetrate dentine tubules; and a pharmaceutical carrier suitable for introduction of the salt MX into a patient.

The nano-sized particles produced in various embodiments of the present invention have many uses in the medical and dental fields, i.e., treatment of dental hypersensitivity, fluoridation to prevent tooth decay and sensitivity, teeth whiteners like carbamide peroxide, and Denclude/Proclude. For example, currently, the only material that is effective for nerve desensitization is potassium nitrate. Due to their small size, use of nanostructured or otherwise nano-sized of potassium nitrate, as found in some embodiments of the invention, enables greatly improved penetration through and transport into micron-size diameter dentinal tubules to reach and directly desensitize the nerves within the dentinal tubules, thus improving performance, potency, and longevity.

In various embodiments, the nano-sized particles of potassium nitrate are between about 10⁻⁶ and 10⁻⁷, 10⁻⁷ and 10⁻⁸, and/or 10⁻⁸ and 10⁻⁹ meters. E.g., in various embodiments, less than 100, 50 or 10 nanometers.

### BRIEF DESCRIPTION OF THE DRAWINGS

FIG. 1 is a flowchart illustrating a generalized method of manufacturing a nano-sized material, according to various embodiments of the invention.

FIG. 2 is a graphic representation of the solubility curves of the salts of one embodiment of the present invention showing moles of solute per 100 g/water verses temperature (°C.).

### DETAILED DESCRIPTION

The present invention utilizes potassium nitrate nanoparticles to increase the effectiveness of nerve desensitization treatments. Dentinal tubules are about a micron in diameter, which is much smaller than state-of-the-art potassium nitrate particles currently in use. The large size of current potassium nitrate particles makes it difficult for them to enter in and transport within the tubules to reach the nerves, thus limiting their overall potential effectiveness. However, the extremely small size of potassium nitrate nanoparticles in this invention (e.g., ≦100 nm, more preferably ≦50 nm, and most preferably ≦10 nm) allow the nanoparticles to more effectively enter into and transport within the dentinal tubules and thus more effectively reach and desensitize the nerves. The small size of the potassium nitrate nanoparticles in this invention enables a larger amount of potassium nitrate to reach the nerves, thus increasing potency. The potassium nitrate nanoparticles in this invention are delivered by incorporation into standard dental products such as toothpastes, gels, solutions, or other delivery methods much in the same fashion as regularly sized potassium nitrate.

### Synthesis of Potassium Nitrate Nanoparticles

### Definitions

“Agitation” refers to the fast or energetic stirring, shaking, or disturbing of a material or substance, especially a liquid substance.

“Coordination species” refers to molecular entities, species, or ligands that coordinate, chelate, bind, or bond to a particle, atom, or molecule. For example, coordinating species are sometimes used to prevent nanoparticles from interacting and aggregating into large clusters.

“Inorganic salt” refers to an inorganic compound comprised of one or more positive cations and one or more negative anions that are bonded together via their electrostatic charges.

“Nano” refers to a size of about 10⁻⁹ meters to about 10⁻⁶ meters.

“Salt” refers to an organic compound comprised of one or more positive cations and one or more negative anions that are bonded together via their electrostatic charges.

“Pressure” refers to a physical force per unit area exerted against a surface. An example of pressure is the force per unit area exerted by a fluid such as a gas or gases, such as that exerted by the atmosphere or that exerted by a gas or gases, or a liquid or liquids.

“Solvent” refers to a fluid, such as a liquid or gas, in which a solute is dissolved to form a solution.

“Temperature” refers to a measure of the average heat or thermal energy of the particles in a substance.

This invention also comprises methods for making nanoparticles (NPs) of potassium nitrate (KNO₃) for the treatment of dentin hypersensitivity. Various synthetic methods are described herein.

### Colloidal Synthesis of KNO₃ NPs

Various embodiments if the invention include a method for the colloidal synthesis of KNO₃ NPs. This method is distinguished from the colloidal synthesis of semiconductor or metal NPs. For example, one distinguishing difference is that KNO₃ is an ionic salt compound whereas semiconductors and metals are often much less ionic in nature. Thus, colloidal synthesis of KNO₃ NPs can require different types of precursors or reactants, reaction types, or conditions relative to semiconductor or metal NPs.

To generate a reasonably narrow size dispersion of semiconductor or metal NPs, reactants or precursors are rapidly reacted in a coordinating solvent in which the coordinating species often function also as soft templates for controlling the formation of nanoparticles in terms of, e.g., its size and shape. These soft templates are often surfactants, trioctylphosphine (TOP), trioctylphosphine oxide (TOPO), micelles, inverse micelles, etc. The reaction is performed rapidly such that products are rapidly created in a non-equilibrium, supersaturated state in the solvent. This supersaturated condition is relieved through the formation or nucleation of seed NPs, thus reducing the concentration of material below that required for nucleation. Reactants (precursors) are optionally then added to the reaction mixture in such a way, e.g., more slowly or adiabatically, that the supersaturated state is not re-generated. Instead, product material is added to the existing seed or nucleated NPs rather than nucleating more seed NPs. This can be achieved, e.g., by creating reaction conditions such that the addition of product material to the seed or nucleated NPs is kinetically the same as or faster than the reaction of reactants to form product material. Thus, further material generated only adds to the existing seed or nucleated NPs. The coordinating species serves as a soft template to restrict or control the growth of the NPs to the desired size, and to cap the surface of the NPs, which in turn passivates the NP surface and stabilizes the resultant colloid by preventing aggregation of the NPs. In general, the higher the concentration of the coordinating species relative to the NPs, the smaller the size of the NPs will be, thus also providing a convenient method to control the NP size.

In the case of the synthesis of colloidal salt NPs with a reasonably narrow size dispersion, such as KNO₃ NPs, the proper conditions of rapid supersaturation in a coordinating solvent followed by the controlled growth of seed or nucleated NPs are achieved through a number of methods not found in the production of semiconductor NPs or metal NPs. One general method is described below.

Unlike the case with semiconductor NPs or metal NPs, the precursors or reactants for salt NPs are other salts that react to give the desired product. As an example, the synthesis of the salt NP MX are achieved via simple displacement reactions of the type in Equation (1) below:

MY+NX→MX+NY

M and N are metallic cations, and X and Y are anions, and the reaction is carried out in a solvent S1.

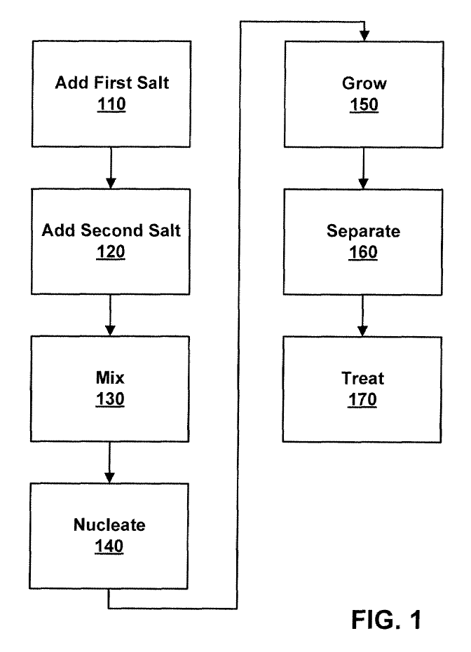

FIG. 1 is a flowchart illustrating a generalized method of manufacturing a nano-sized material, according to various embodiments of the invention. In this method a desired non-equilibrium supersaturation state of a salt MX in solvent S1 is generated using reactant salts (precursors) MY and NX and solvent S1. Nano-sized MX is the desired nano-sized material. The salts are selected such that the solubilities of MY, NX and NY in S1 are larger than the solubility of MX in S1 under the reaction conditions or parameters (such as temperature, pressure, reactant (precursor) and product concentrations, type and concentration of coordinating species, such as surfactants, etc.).

More specifically in an Add First Salt Step 110, the salt MY is added to a solvent S1. In an Add Second Salt Step 120 the salt NX is added to the solvent S1. The Salt NX may be added as a solid or as a pre-solvated component of a second solution. In a Mix Step 130, quantities and/or solutions (in the appropriate concentrations) of MY and NX are rapidly, or non-adiabatically, mixed in the solvent S1 containing soft templates (one skilled in the art will recognize that a variety of soft template, such as coordinating species, can be used for this purpose such as surfactants, Triton X, glycols, oleic acid, TOPO, micelles, inverse micelles, etc. to yield a coordinating solvent) such that the resultant product MX is rapidly produced in a non-equilibrium, supersaturated state (due to the rapid kinetics) such as via a stoichiometric displacement reaction.

In a Nucleate Step 140, the supersaturated state is relieved through the nucleation of MX seed NPs, controlled in part by the soft template (e.g., surfactants, micelles, inverse micelles, etc.). Nucleation may be spontaneous or triggered by addition of a nucleation reagent.

In an optional Grow Step 150, growth of the seed NPs to their desired size is then achieved through the further addition of MY and NX, optionally in a fashion such that the supersaturated state is not re-generated. This can be done in an adiabatic fashion by controlling the amount and addition rate of reactants (precursors) and other reaction parameters such as temperature, such that the addition of product MX material to existing nucleated MX NPs is faster than the reaction of MY and NX to form MX. In this way, further MX material produced is added to the seed NPs, growing these NPs, rather than generating more seed NPs.

In a Separate Step 160, the salt MX is separated from the solution S1. This may be performed, for example, by centrifugation, filtration, ultra filtration, dialysis, combustion thereof, or the like.

In an optional Treatment Step 170, the salt MX is used for medical treatment. For example, in some embodiments the MX salt is used to treat dentin hypersensitivity. In these embodiments, the particle size of the MX salt may be selected in Grow Step 150 to enter dentinal tubules. Treatment may include, for example, applying the MX salt to the dentine using an appropriate pharmaceutical carrier, (e.g., powder, emulsion, suspension, gel, liquid, paste or combination thereof), and then allowing the salt to penetrate the dentin tubules.

In the case with semiconductor or metal NPs, the reaction kinetics are determined by the kinetics of decomposition or thermolysis of the precursors, their reaction, and the subsequent formation of product. The decomposition of the precursors is often an endothermic process, requiring heat to complete, thus the reactions are often performed at high temperatures. When the NPs reach the desired size, the reaction is stopped by lowering the reaction temperature, thus stopping the endothermic reaction.

However, in the case with salt NPs, the reaction is essentially a solvation reaction followed by a diffusion process in which the solvated ions react to form products. The reaction kinetics are determined by the rate of solvation of the salt reactants (to yield cations and anions) and the rate of diffusion of these ions to each other to form the salt product. This is often rapid and requires little thermal energy (small reaction barrier). Therefore, the reaction is often carried out at lower temperatures than that required for the synthesis of semiconductor or metal NPs. Furthermore, the reactant salts are optionally pre-solvated or dissolved in a solvent before mixing and reacting the two solutions together. This eliminates the solvation step and leaves diffusion of the reactant ions to determine the reaction rate. Thus, the temperature only needs to be sufficiently high to foster rapid diffusion of the solvated ions. Therefore, these reactions are conducted at lower temperatures, and when the NPs reach the desired size, equation (1) is stopped by simply halting the further addition of reactants (precursors), though other methods such as lowering the temperature also works by rapidly slowing the solvation and/or diffusion rate.

One consideration in selecting or optimizing the appropriate reaction parameters is to optimize or maximize the yield of the reaction, in some embodiments, the reaction parameters are chosen so that there is the largest difference in the solubility of the product MX and the least soluble of the reactants (precursors MY and NX) or by-products (NY), without hindering the desired reaction (1). Of course, the solubility of MX is less than the solubility of the reactants (MY and NX) or by-product (NY). In this way, the degree of supersaturation of MX is the largest without supersaturating or precipitating out the other materials and resulting in the formation of NPs of these other materials (unless this is desired). This generates a larger or the largest number or amount of MX NPs. For example, if the reaction parameter to optimize is the reaction temperature, then the reaction temperature is chosen such that there is the largest difference in solubility between MX and the least soluble material within the group MY, NX, and NY. Generally, if the solubility increases monotonically with temperature (which is usually the case), this will entail running the reaction at the lowest temperature possible or reasonable without unduly hindering the reaction or reaction kinetics such as the kinetics of solvation and/or diffusion.

Various embodiments of this invention are described by the discussion of reaction (1) is illustrated by the colloidal synthesis of KNO₃ NPs. In this case, reactants supply K⁺ ions and NO₃⁻ ions. Examples of such reactants for supplying K⁺ions are: KCl, KI, K₂SO₄, KOH. Examples of such reactants for supplying NO₃⁻ ions are: NaNO₃, NH₄NO₃, AgNO₃, Ca(NO₃)₂. One skilled in the art will also recognize other sources of K⁺ ions and NO₃⁻ ions. In addition, a coordinating solvent is optionally used and this is accomplished by using a soft template or surfactants, with examples including, but not limited to, Triton X-100, glycols, oleic acid, micelles, inverse micelles, TOPO, and the like.

### EXAMPLE 1

### Reaction of KCl and NaNO₃

In this example, the reactants KCl and NaNO₃ are used, and the relevant reaction to synthesize KNO₃ NPs is:

KCl+NaNO₃→KNO₃ (Colloidal NPs)+NaCl (2)

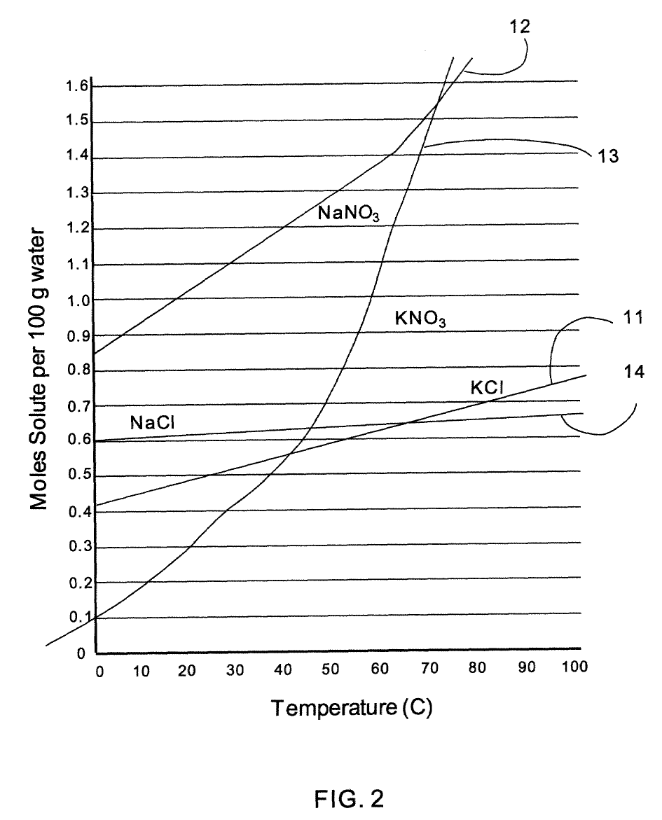

In this case, the reaction is carried out in aqueous conditions since the solubility (moles of solute/100 g H₂O) of KCl, NaNO₃, and NaCl in water are all greater than KNO₃ through a broad temperature range (less than about 39° C.), as shown in FIG. 2, with curves KCl (11), NaNO₃ (12), KNO₃ (13) and NaCl (14). With regard to the solubility of the products, KNO₃ is less soluble (moles of solute/100 g H₂O) than NaCl over an even broader temperature range (less than about 44° C.). The result of this reaction as described above is the generation of colloidal NPs of KNO₃ along with solvated ions. One specific embodiment of this synthesis is the following:

The reaction is run at 0° C., though it will run at any temperature within the range specified herein, including less than about 45, 40, 35, 30, 25, 20, 15, 10, 5 or 0° C. The stoichiometrically limiting reactant is made to be KCl due to its relatively lower solubility. From FIG. 2, a saturated aqueous solution of KCl at 0° C. is made with a concentration of about 0.41 moles of KCl/100 g of H₂O. A stoichiometrically equivalent amount of NaNO₃ is prepared in a solution with a concentration of about 0.41 moles of NaNO₃/50 g H₂O at 0° C. From FIG. 2, it is clear that this NaNO₃ solution is not saturated. A coordinating species or surfactant such as Triton X-100 is added into either the KCl or NaNO₃ solution in the amount of roughly one drop per milliliter of solution. With vigorous stirring of the solution with the coordinating species, the other solution is rapidly added to the stirred solution, maintaining a temperature of 0° C. This rapidly creates KNO₃ and NaCl in the effective concentration of about 0.27 moles of KNO₃/100 g H₂O and about 0.27 moles of NaCl/100 g H₂O. Since the saturation concentration of NaCl is about 0.6 moles/100 g H₂O at 0° C., the NaCl remains in solution. However, since the saturation concentration of KNO₃ at 0° C. is about 0.1 moles of KNO₃/100 g H₂O, this results in a rapidly generated supersaturated state of KNO₃. NPs of KNO₃ are thus created to relieve this supersaturated state. To grow the NPs in size, additional NaNO₃ and KCl are slowly added in stoichiometric quantities with vigorous stirring until the desired NP size is achieved, the reaction. The nano sized KNO₃ particles are then separated from the other reactants and products by centrifugation, filtration, ultra filtration, dialysis and combustion thereof.

### EXAMPLE 2

### KNO₃ Formation at About 16° C. in Nanoparticle Size

Some embodiments of this synthetic method for KNO₃ NPs include the following:

The reaction is first run at any temperature above approximately 16° C. For example, between 45 and 16° C. This temperature is T1 ° C. The stoichiometric limiting reactant is again made to be KCl due to its low solubility. An aqueous solution of KCl at temperature T1° C. is made with a concentration of about 0.41 moles of KCl/100 g or H₂O. A stoichiometric equivalent amount of NaNO₃ is prepared in a solution with a concentration of about 0.41 moles of NaNO₃/50 g H₂O at T=T1 ° C. A coordinating species or surfactant such as Triton X-100 is added into either the KCl or NaNO₃ solution in the amount of roughly one drop per milliliter of solution. With vigorous stirring of the solution with the coordinating species, the other solution is added to the stirred solution, maintaining a temperature of above about 16° C. such as T1 ° C. This creates KNO₃ and NaCl in the effective concentration of about 0.27 moles of KNO₃/100 g H₂O and about 0.27 moles of NaCl/100 g H₂O. These concentrations are not above the saturated state for any of the products, provided that T1 is above about 16° C. for KNO₃. The solution is stirred vigorously for a few minutes (e.g. about 5 or 10 minute), to about an hour at a temperature above about 16° C. or T1 ° C. Then, the solution is rapidly cooled to about 0° C. (or any temperature less than about 16° C. since the saturation concentration of KNO₃ at 16° C. is about 0.27 moles/100 g H₂O). This rapidly generates KNO₃ in a supersaturated state, but the other salts remain below their saturation state. NPs of KNO₃ are thus created to relieve this supersaturated state. To grow the NPs in size, additional NaNO₃ and KCl are slowly added in stoichiometric quantities with vigorous stirring until the desired NP size is achieved. When the desired NP size is achieved, the nano sized KNO₃ particles are then separated from the other reactants and products by centrifugation, filtration, ultra filtration, dialysis or combinations thereof.

### EXAMPLE 3

### KNO₃ Formation at About 50° C. in Nanoparticle Size

Some embodiments of this synthetic method include the following: The reaction is first run at a temperature above approximately 50° C. Let this temperature be T1 ° C. An aqueous solution of KCl at temperature T1 ° C. is made with a concentration of about 0.6 moles of KCl/100 g or H₂O. A stoichiometric equivalent amount of NaNO₃ is prepared in a solution with a concentration of about 0.6 moles of NaNO₃/46 g H₂O at T=T1 ° C. A coordinating species or surfactant such as Triton X-100 is added into either the KCl or NaNO₃ solution in the amount of roughly 1 drop per milliliter of solution. With vigorous stirring of the solution with the coordinating species, the other solution is added to the stirred solution, maintaining a temperature of above about 50° C. such as T1 ° C. This creates KNO₃ and NaCl in the effective concentration of 0.41 moles of KNO₃/100 g H₂O and 0.41 moles of NaCl/100 g H₂O. These concentrations are below the saturation concentration for any of the products, provided that T1 is above about 50° C. The solution is stirred vigorously for about a few minutes to about an hour at a temperature above about 50° C. or T1 ° C. Then, the solution is rapidly cooled to about 0° C. (or any temperature less than about 27° C. since the saturation concentration of KNO₃ at about 28° C. is about 0.41 moles/100 g H₂O). This rapidly generates KNO₃ in a supersaturated state (higher than in example 2), but the other salts remain below their saturation state. NPs of KNO₃ are thus created to relieve this supersaturated state. To grow the NPs in size, additional NaNO₃ and KCl are slowly added in stoichiometric quantities with vigorous stirring until the desired NP size is achieved. When the desired NP size is achieved, the nano sized KNO₃ particles are then separated from the other reactants and products by centrifugation, filtration, ultra filtration, dialysis or combinations thereof.

### EXAMPLE 4

### KNO Formation at About 50° C. in Nanoparticle Size

Some embodiment of this synthetic method include the following:

The reaction is first run at a temperature above approximately 50° C. Let this temperature be T1 ° C. An aqueous solution of KCl at temperature T1 ° C. is made with a concentration of about 0.6 moles of KCl/100 g of H₂O. Into the KCl solution is added a coordinating species or surfactant such as Triton X-100 in the amount of roughly 1 drop per milliliter of solution. With vigorous stirring of the KCl solution, a stoichiometric amount of NaNO₃ is added to the KCl solution, maintaining a temperature of greater than about 50° C. such as T1 ° C. This creates KNO₃ and NaCl in the effective concentration of about 0.6 moles of KNO₃/100 g H₂O and about 0.6 moles of NaCl/100 g H₂O. These concentrations are below the saturation concentration for any of the products, provided that the temperature is above about 50° C. The solution is stirred vigorously for about a few minutes to about an hour at a temperature above about 50° C. or T1 ° C. Then, the solution is rapidly cooled to 0° C. (or any temperature less than about 42° C. since the saturation concentration of KNO₃ at about 42° C. is about 0.6 moles/100 g H₂O). This rapidly generates KNO₃ in a supersaturated state (higher than in Examples 2 and 3), but the other salts remain below their saturation state. NPs of KNO₃ are thus created to relieve this supersaturated state. To grow the NPs in size, additional NaNO₃ and KCl are slowly added in stoichiometric quantities with vigorous stirring until the desired NP size is achieved. When the desired NP size is achieved, the nano sized KNO₃ particles are then separated from the other reactants and products by centrifugation, filtration, ultra filtration, dialysis or combinations thereof.

Other example reactions for the synthesis of KNO₃ nanoparticles using the concepts and methods in the above embodiments can be performed using different reactants that also supply K⁺ and NO₃⁻ ions. Examples of reactions using some of these other reactants are given in reactions (3) and (4) below.

KCl+NH₄NO₃→KNO₃ (Colloidal NPs)+NH₄Cl (3)

KI+NaNO₃→KNO₃ (Colloidal NPs)+NaI (4)

One skilled in the art can envision other reactants that can supply the requisite K⁺ and NO₃⁻ ions, and thus carry out the synthesis using these reactants in the general manner described above. These other reactants are included herein as embodiments of this invention.

Another type of reaction that can be used is one where one of the reaction products precipitates out of solution, leaving the desired product as colloidal NPs. The reaction is then driven forward by Le Chatelier's Principle. Therefore, the reaction is performed as in the above embodiments in which the reactants are rapidly mixed and/or cooled in a non-adiabatic fashion to generate a non-equilibrium, supersaturated state. The NPs are then grown by further adding reactants in an adiabatic fashion such that product materials formed are added to existing nucleated NPs rather than nucleating more NPs. In addition, the insoluble product precipitates out of solution, driving the reaction further. When the desired NP size is reached, since the desired colloidal NPs are left in solution, the precipitate is filtered out, leaving the colloidal suspension of NPs. Examples of this type of reaction are given below.

### EXAMPLE 5

### KCl+AgNO₃ at °C.

One embodiment of this is given in the following reaction:

KCl+AgNO₃→KNO₃ (Colloidal NPs)+AgCl₍ₛ₎ (5)

As in the above embodiments, the reactants are very quickly mixed in a coordinating solvent to rapidly create a non-equilibrium supersaturated state of KNO₃. In this case, the AgCl, which has a very low solubility in water, precipitates out and drives the reaction forward, and colloidal NPs of KNO₃ are nucleated. The NP size can be grown by adding more reactants in the manner described in the above embodiments, with the key condition being not to re-generate the non-equilibrium supersaturated state while growing the NPs to the desired size. When the reaction is completed, the AgCl precipitate is filtered and the supernatant containing the colloidal KNO₃ NPs is collected.

Other embodiments of this type of reaction are given by the use of other reactants that generate a solid precipitate along with KNO₃, as illustrated in the following reactions:

K₂SO₄+Ca(NO₃)₂→2KNO₃ (Colloidal NPs)+CaSO₄₍ₛ₎ (5)

2KCl+Ca(NO₃)₂+2KNO₃ (Colloidal NPs)+CaCl₂₍ₛ₎ (5)

Both CaSO₄ and CaCl₂ are insoluble in water and will precipitate out, leaving a colloidal suspension of KNO₃ NPs, as described in Example 1-4 above.

The above synthetic methods of this invention are meant to generate NPs. However, there are circumstances where the size dispersion of the salt NPs is not required to be small, as may be in the case of KNO₃ NPs for the treatment of dentin hypersensitivity, provided the KNO₃ NPs are smaller than the diameter of the dentinal tubules. Any size nano particle is produced in a controlled process. The size is controlled by the dispersion components, speed of reagent addition, time, temperature, agitation, etc. In these cases, the reaction can be simplified, as discussed below.

For example, the rapid creation of a non-equilibrium, supersaturated state to nucleate NPs followed by growth of these seed NPs, which is needed to generate NPs with a small size dispersion, may not be required. Instead, the reactants can be reacted, e.g., in an adiabatic fashion, by mixing them in a slower rate under the appropriate reaction conditions (e.g., appropriate proportions and concentrations of reactants, temperature, solvent, etc.) such that the desired product is produced in a state above the saturation concentration (rather than a supersaturated state) to precipitate a colloidal suspension of NPs, and this state need not be generated in a rapid fashion. Further or continued addition of reactants serve to both nucleate more NPs and grow existing NPs, thus leading to a larger size dispersion.

In addition, the use of coordinating species in the initial reaction mixture is optional, and if coordinating species are used, coordinating species could be added to the reaction mixture after the reaction is completed and NPs are formed to prevent their aggregation. As an example, the reactants can be mixed normally in a coordinating solvent, where the coordinating species help to seed NPs from the mixture. In this case, the further or continued addition of reactants serve to both nucleate more NPs and grow existing NPs, thus leading to a larger size dispersion.

The reactants are also mixed normally in a solvent without coordinating species. NPs are still nucleated from a solution without coordinating species, though it may be less efficient or effective and may lead to some fraction of larger or bulk particles. After the formation of NPs, coordinating species are added to the resulting colloidal suspension of NPs. These coordinating species coordinate to the surface of the NPs and stabilize the colloidal suspension and prevent aggregation of the NPs.

Embodiments of this type of synthetic method, where the size dispersion is larger, are variations of the method and examples (Examples 1-5) described above where the size dispersion is smaller. The critical difference is that the reactants do not have to be rapidly mixed to create a supersaturated state. Rather, they are slowly or adiabatically added to each other. In addition, coordinating species are or are not used. as described above for this larger size dispersion synthetic method. Other than these difference, embodiments of these larger size dispersion methods are the same as embodiments for the smaller size dispersion methods.

Specific embodiments of this larger size dispersion method are described below:

### EXAMPLE 6

### KCl and NaNO₃

In a quantity of boiling water, add KCl until a concentration that is approximately about 0.6 moles of KCl/100 g of H₂O is attained. Next, add a stoichiometric equivalent of NaNO₃ to boiling H₂O until a concentration is reached in the range of about 1.2 moles of NaNO₃/100 g of H₂O to 2 moles of NaNO3/100 g of H₂O. Stir until all NaNO3 is dissolved. Then add to the NaNO₃ solution a quantity of a coordinating species such as Triton X-100®, in the amount of about one drop/milliliter of solution, and continue to boil the solution. The Triton X-100® may also or instead be added to the KCl solution. With vigorous stirring of the boiling solution with the Triton X-100® added, which we will take for this example to be the NaNO₃ solution, slowly add (e.g., dropwise) the other solution, which we will take for this example to be the KCl solution. Continue stirring for a few minutes to an hour. Depending on the concentration of the KCl, NaCl may precipitate out of solution. If a precipitate is formed, the precipitate may be removed. Slowly lower the temperature of the solution. The temperature can be lowered to any temperature that is below about 42° C. (since about 42° C. is the temperature at which the saturation concentration of KNO₃ in moles of KNO₃/100 g of H₂O is about the same as the initial concentration of KCl in moles of KCl/100 g of H₂O, i.e., about 0.6 moles of KC/100 g of H₂O), but room temperature (about 25° C.) and the freezing point of water (about 0° C.) are convenient temperatures. As the solution cools, NaCl may precipitate out. Again, this precipitate may be removed. When the final temperature is reached, filter out the precipitate. The remaining supernatant contains a colloidal suspension of KNO₃ NPs. Add more Triton X-100® to make the final concentration of Triton X-100® be about one drop/milliliter of final solution.

### EXAMPLE 7

### KCl and NaNO₃

In a quantity of boiling water, add KCl until a concentration that is about 0.6 moles of KCl/100 g of H₂O is attained. Then add to the KCl solution a quantity of a coordinating species such as Triton X-100® in the amount of about 1 drop/milliliter of solution, and continue to boil the solution. Next, with vigorous stirring, slowly add a stoichiometric equivalent of NaNO₃ to the boiling KCl solution. (An initial solution of NaNO₃ in the approximate concentration of about 0.6 moles of NaNO₃/100 g of H₂O may be prepared in boiling water instead of a solution of KCl. In this case, the Triton X-100® is added to the NaNO₃ solution and an equivalent amount of KCl is added to the boiling NaNO₃ solution. In this example, we will assume that the initial solution is of KCl.) Continue stirring for a few minutes to an hour. Depending on the concentration of the KCl, NaCl may precipitate out of solution. If a precipitate is formed, the precipitate may be removed. Slowly lower the temperature of the solution. The temperature can be lowered to any temperature that is below about 42° C. (since about 42° C. is the temperature at which the saturation concentration of KNO₃ in moles of KNO₃/100 g of H₂O is about the same as the initial concentration of KCl in moles of KCl/100 g of H₂O, i.e., about 0.6 moles of KCl/100 g of H₂O), but room temperature (about 25° C.) and the freezing point of water (about 0° C.) are convenient temperatures. As the solution cools, NaCl may precipitate out. Again, this precipitate may be removed. When the final temperature is reached, filter out the precipitate. The remaining supernatant contains a colloidal suspension of KNO₃ NPs. Add more Triton X-100® to make the final concentration of Triton X-100® be about 1 drop/milliliter of final solution.

### EXAMPLE 8

In a quantity of water at a temperature of about room temperature (about 25° C.), add NaNO₃ until a concentration that is approximately about 0.6 moles of NaNO₃/100 g of H₂0 is attained. Then add to the NaNO₃ solution a quantity of a coordinating species such as Triton X-100 ® in the amount of about 1 drop/milliliter of solution. Next, with vigorous stirring, slowly add a stoichiometric equivalent of KCl to the NaNO₃ solution. Continue stirring for a few minutes to an hour. Depending on the concentration of the NaNO₃, NaCl precipitates out of solution. If a precipitate is formed, the precipitate is removed. At this point, a colloidal suspension of KNO₃ NPs is formed. More KNO₃ NPs may be generated if the temperature is lowered. In this case, slowly lower the temperature of the solution. The temperature is lowered to any temperature below room temperature, but the freezing point of water (about 0° C.) is a convenient temperature. As the solution cools, NaCl may precipitate out. Again, this precipitate, if present, is removed. When the final temperature is reached, filter out the precipitate. The remaining supernatant contains a colloidal suspension of KNO₃ NPs. Add more Triton X-100® to make the final concentration of Triton X-100® be about 1 drop/milliliter of final solution.

### EXAMPLE 9

### KCl and NaNO₃ at 25° C.

In a quantity of water at a temperature of about room temperature (about 25° C.), add KCl until a concentration that is about 0.51 moles of KCl/100 g of H₂O is attained. Then add to the KCl solution a quantity of a coordinating species such as Triton X-100® in the amount of about 1 drop/milliliter of solution. Next, with vigorous stirring, slowly add a stoichiometric equivalent of NaNO₃ to the KCl solution. Continue stirring for a few minutes to an hour. Depending on the concentration of the KCl, NaCl precipitates out of solution. If a precipitate is formed, then the precipitate may be removed. At this point, a colloidal suspension of KNO₃ NPs is formed. More KNO₃ NPs may be generated if the temperature is lowered. In this case, slowly lower the temperature of the solution. The temperature can be lowered to any temperature below room temperature, but the freezing point of water (about 0° C.) is a convenient temperature. As the solution cools, NaCl may precipitate out. Again, this precipitate may be removed. When the final temperature is reached, filter out the precipitate. The remaining supernatant contains a colloidal suspension of KNO₃NPs. Add more Triton X-100® to make the final concentration of Triton X-100® be about 1 drop/milliliter of final solution.

There are many variations of all these methods or guiding principles described herein for synthesizing KNO₃ NPs. There are variations in the types of reactant compounds (such as other compounds that supply K⁺ ions and NO₃⁻ ions), reaction temperatures, reactant concentrations, methods for mixing the reactants, methods for generating a non-equilibrium supersaturated state, reaction times, and so forth. One skilled in the art will readily recognize and anticipate these variations, which are included in this patent herein.

In addition, it is possible that the methods used for the optimization or tailoring of the properties of semiconductor or metal NPs are also adapted to the case for salt NPs, taking into consideration the principles outlined herein to account for the differences between salt NPs and semiconductor and metal NPs. One skilled in the art will readily recognize and anticipate these variations, which are included in this patent herein.

### EXAMPLE 10

Synthesis of KNO₃ Nanoparticles with Ball Milling Techniques

Another embodiment of this invention is the use of ball milling techniques to fabricate nanoparticles of KNO₃. This includes conventional ball milling and high-energy ball milling. Ball milling can be used as a mechanical or mechanochemical method to fabricate nano-sized materials and has been used to generate nano-sized materials from a variety of materials such as semiconductors, metals, metal oxides, etc. However, it has not been recognized that conventional ball milling and high-energy ball milling can be used for fabricating nanoparticles of KNO₃, which is included in this invention herein.

Conventional ball milling typically yields particles on the order of roughly a micron. However, high-energy ball milling can produce particles on the order of less than 100 nm or more preferably less than 50 nm.

In addition, various reactants such as described above to provide K⁺ ions and NO₃⁻ ions can be placed in a ball mill where mechanochemical methods can be used to fabricate nanoparticles of KNO₃.

Several embodiments are specifically illustrated and/or described herein. However, it will be appreciated that modifications and variations are covered by the above teachings and within the scope of the appended claims without departing from the spirit and intended scope thereof. For example, a further advancement in treatment for hypersensitivity is to incorporate two active ingredients in which one penetrates the tubule to desensitize the nerve while the other occludes the tubule. It is also possible for the same agent to perform both of these two functions. For example, a mixture of potassium nitrate of different sizes.

The embodiments discussed herein are illustrative of the present invention. As these embodiments of the present invention are described with reference to illustrations, various modifications or adaptations of the methods and or specific structures described may become apparent to those skilled in the art. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

I claim:

1. A method for the production of nano-sized particles of a salt MX, the method comprising:
mixing reactant salts MY and NX in a solvent S1, in the presence of a soft template, chosen such that the solubilities of salts MY, NX and NY in S1 are larger than the solubility of salt MX in S1 at a reaction temperature;
forming the nano-sized particles of salt MX by nucleation under supersaturated conditions; and
separating the nano-sized particles of salt MX from the solvent.

2. The method of claim 1, wherein the salt NX is solvated separately from the salt MY.

3. The method of claim 1, wherein the salts MY and NX are first mixed in the solvent under non-adiabatic conditions, and further comprising adding more MY or NZ to the solvent under adiabatic conditions to grow the formed nano-sized salt particles of MX.

4. The method of claim 1, wherein the nano-sized particles of salt MX comprise potassium nitrate, sodium monofluorophosphate (MFP), acidulated phosphate fluoride (APF), stannous fluoride, tin(II) fluoride (SnF2), amine fluorides (e.g., OLAFLUR® (N′-octadecyltrimethylenediamine-N,N,N′-tris(2-ethanol)-dihydrofluoride), DECTAFLUR® (9-octadecenylamine-hydrofluoride)), calcium fluoride, or a combination thereof.

5. A method for the size controlled production of nano-sized salt particles, the method comprising:
a) adding a salt MY to a solvent comprising NX and a coordinating species, under non-adiabatic conditions;
b) mixing the resulting solution to generate a supersaturated solution of salt MX and NY,
c) maintaining said solution under non-adiabatic conditions to promote the nucleation of seed nano-sized particles of MX;
d) increasing the size of the seed nano-sized particles of MX to a specific size range by further controlled addition of MY and NX under adiabatic conditions; and
e) stopping the addition of MY or NX so as to stop the growth of the formed nanoparticles of MX; and
f) separating the nano-sized particles of MX having a specific size range from N ions or Y ions, wherein M is a first organic or inorganic cation, Y is a first anion, N is a second different organic or inorganic cation, and X is a second different anion.

6. The method of claim 5, wherein the coordinating species is selected from the group consisting of surfactants micelles and inverse micelles.

7. The method of claim 5, wherein increasing the size of the seed nano-sized particles of MX is performed without creating a second supersaturated state.

8. The method of claim 5, wherein mixing the resulting solution is performed at a temperature less than about 45° C.

9. The method of claims 5, further including introducing the nano-sized particles of MX into a pharmaceutically acceptable carrier configured for dental use.

10. A method of treating dentin sensitivity, which method comprises:
applying to the dentin nano-sized particles in a therapeutically effective amount in a pharmaceutically acceptable carrier; and
allowing the nano-sized particles to penetrate tubules of the dentine.

11. The method of claim 10, wherein the nano-sized particles are chosen from among potassium nitrate, sodium monofluorophosphate (MFP), acidulated phosphate fluoride (APF), stannous fluoride, tin(II) fluoride (SnF2), amine fluorides (e.g., OLAFLUR® (N′-octadecyltrimethylenediamine-N,N,N′-tris(2-ethanol)-dihydrofluoride), DECTAFLUR® (9-octadecenylamine-hydrofluoride)), calcium fluoride, or combination thereof.

12. The method of claim 10, wherein the nano-sized particles include potassium nitrate.

13. The method of claim 10, wherein the therapeutically effective amount is between about 0.001 and 1 mg.

14. The method of claim 10, wherein the pharmaceutically acceptable carrier includes a powder, emulsion, suspension, gel, liquid, paste or combination thereof.

15. The method of claim 10 wherein the nano-sized particles are in a size between 10⁻⁹ and 10⁻⁸ meters.

16. The method of claim 10 wherein the nano-sized particles are in a size between 10⁻⁸ and 10⁻⁷ meters.

17. The method of claim 10 wherein the nano-sized particles are in a size between 10⁻⁷ and 10⁻⁶ meters.

18. A composition for the production of nano-sized particles, which composition comprises:
a supersaturated solution of a salt MX, M including an organic or inorganic cation non-adiabatically introduced to the solution as a salt MY, X including an organic or inorganic anion non-adiabatically introduced to the solution as a salt NX, the supersaturated solution being at a temperature of less than about 45° C.; and
a soft template configured for controlling the formation of nano-sized particles of the salt MX.

19. The composition of claim 18, wherein MX includes potassium nitrate.

20. A composition comprising:
nano-sized particle of a salt MX configured to penetrate dentine tubules; and
a pharmaceutical carrier suitable for introduction of the salt MX into a patient.

21. The composition of claim 20, wherein the nano-sized particles are chosen from among potassium nitrate, sodium monofluorophosphate (MFP), acidulated phosphate fluoride (APF), stannous fluoride, tin(II) fluoride (SnF2), amine fluorides (e.g., OLAFLUR® (N′-octadecyltrimethylenediamine-N,N,N′- tris(2-ethanol)-dihydrofluoride), DECTAFLUR® (9-octadecenylamine-hydrofluoride)), calcium fluoride, or combination thereof.

22. The composition of claim 20, wherein the nano-sized particles comprise potassium nitrate.

23. The method of claim 210 wherein the nano-sized particles are in a size between 10⁻⁹ and 10⁻⁷ meters.
